(19) United States
(12) Patent Application Publication
(10) Pub. No.: US 2025/0135279 A1
(43) Pub. Date: May 1, 2025

(54) GOLF CLUB RECOMMENDATION SERVICE DEVICE

(71) Applicant: RNDUS CO., LTD., Seoul (KR)

(72) Inventor: KYUSHIK JUNG, Seongnam-si (KR)

(73) Assignee: RNDUS CO., LTD., Seoul (KR)

(21) Appl. No.: 18/812,417

(22) Filed: Aug. 22, 2024

(30) Foreign Application Priority Data
Oct. 26, 2023 (KR) 10-2023-0144932

(51) Int. Cl. A63B 24/00; A63B 69/36

(52) U.S. Cl. CPC A63B 24/0003; A63B 69/3605; A63B 2220/30

(57) ABSTRACT

A golf club recommendation service device includes a shot information acquisition unit acquiring club-specific shot information including ball measurement data and club measurement data during a ball impact process of a user; a target distance reception unit receiving a target distance constituted by horizontal and vertical distances from a current location up to a target location; and a club recommendation unit recommending a specific club by analyzing the club-specific shot information of the user based on the target distance.

### CROSS-REFERENCE TO PRIOR APPLICATION

This application claims priority to Korean Patent Application No. 10-2023-0144932 (filed on Oct. 26, 2023), which is hereby incorporated by reference in its entirety.

### BACKGROUND

The present disclosure relates to a golf club recommendation service technology, and more particularly, to a golf club recommendation service device which can recommend a personalized club suitable for a distance up to a target measured based on average shot data for each club of a user.

In recent years, as golf facilities such as screen golf courses and indoor golf practice ranges have increased, golf is gradually developing as one of popular sports in Korea. In particular, as the golf population increases, the number of golfers in the field is increasing.

When a golfer performs rounding on a field, the golfer sends a golf ball away, and then moves to a location where the golf ball falls by taking a cart. The golfer approximately estimates distances up to the golf balls and a target to select a specific club among various clubs in the cart, and then walks and moves up to the golf ball with the club.

However, if the golf ball is far from the location of the cart, it is not known in detail the distance and a method of attack from the golf ball to the target, so it is common that the golfer moves to the location of the golf ball with two or three clubs through the guess.

Therefore, a technique can be required, which can check the exact information about the distance from the golf ball or the distance from the golf to the target, and provide recommendation information about a club according to the distance or a club that meets an individual play level.
Related Art: Korean Patent Registration No. 10-2242994 (Apr. 15, 2021)

### SUMMARY

In view of the above, an embodiment of the present disclosure provides a golf club recommendation service device which can recommend a personalized club suitable for a distance up to a target measured based on average shot data for each club of a user.

Among embodiments, a golf club recommendation service device includes: a shot information acquisition unit acquiring club-specific shot information including ball measurement data and club measurement data during a ball impact process of a user; a target distance reception unit receiving a target distance constituted by horizontal and vertical distances from a current location up to a target location; and a club recommendation unit recommending a specific club by analyzing the club-specific shot information of the user based on the target distance.

The shot information acquisition unit may acquire club information of the user, and receive at least one of a ball speed, a launch angle, and a flight distance of a ball measured during the ball impact process as the ball measurement data.

When at least some data of the ball speed, the launch angle, and the flight distance are not acquired, the shot information acquisition unit may estimate some data not acquired with the remaining data.

The shot information acquisition unit may acquire the club information of the user, and receive at least one of a club speed, a smash factor, and an attack angle of a club ball measured during the ball impact process as the club measurement data.

The shot information acquisition unit may load the club-specific shot information measured when the user practices in an indoor golf practice range.

The target distance reception unit may calculate the horizontal and vertical distances based on measurement information including a straight distance and a slope up to the target location.

The club recommendation unit may determine N (the N is a natural number) candidate clubs having a flight distance closest to the horizontal distance, and recommend one candidate club having a smallest measurement deviation of the club-specific shot information among the N candidate clubs.

The club recommendation unit may calculate an additional distance up to the target location based on the vertical distance to determine a shot recommendation distance based on the horizontal distance and the additional distance, and analyze the club-specific shot information based on the shot recommendation distance to recommend a specific club.

The disclosed technology may have the following effects. However, since it is not meant that a specific embodiment should include all of the following effects or merely include the following effects, the scope of the disclosed technology is not to be construed as being limited thereby.

According to an embodiment of the present disclosure, the golf club recommendation service device can recommend a personalized club suitable for a distance up to a target measured based on average shot data for each club of a user.

### BRIEF DESCRIPTION OF THE DRAWINGS

FIG. 1 is a diagram describing a recommendation service system according to the present disclosure.

FIG. 2 is a diagram describing a system configuration of a recommendation terminal of FIG. 1.

FIG. 3 is a diagram describing a functional configuration of the recommendation terminal of FIG. 1.

FIG. 4 is a flowchart describing an embodiment of a golf club recommendation service process according to the present disclosure.

FIG. 5 is a diagram describing an embodiment of a process of calculating horizontal distances up to a ball and a target according to the present disclosure.

FIG. 6 is a diagram describing an embodiment of a process of calculating a shot recommendation distance for sending the ball to the target according to the present disclosure.

FIG. 7 is a diagram for describing an embodiment of shot information for each club of a user according to the present disclosure.

### DETAILED DESCRIPTION

A description of the present disclosure is merely an embodiment for a structural or functional description and the scope of the present disclosure should not be construed as being limited by an embodiments described in a text. That is, since the embodiment can be variously changed and have various forms, the scope of the present disclosure should be understood to include equivalents capable of realizing the technical spirit. Further, it should be understood that since a specific embodiment should include all objects or effects or include only the effect, the scope of the present disclosure is limited by the object or effect.

Meanwhile, meanings of terms described in the present application should be understood as follows.

The terms “first,” “second,”, and the like are used to differentiate a certain component from other components, but the scope of should not be construed to be limited by the terms. For example, a first component may be referred to as second component, and similarly, the second component may be referred to as first component.

It should be understood that, when it is described that a component is “connected to” another component, the component may be directly connected to another component or a third component may be present therebetween. In contrast, it should be understood that, when it is described that an element is “directly connected to” another element, it is understood that no element is present between the element and another element. Meanwhile, other expressions describing the relationship of the components, that is, expressions such as “between” and “directly between” or “adjacent to” and “directly adjacent to” should be similarly interpreted.

It is to be understood that the singular expression encompass a plurality of expressions unless the context clearly dictates otherwise and it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

In each step, reference numerals (e.g., a, b, c, etc.) are used for convenience of description, the reference numerals are not used to describe the order of the steps and unless otherwise stated, it may occur differently from the order specified. That is, the respective steps may be performed similarly to the specified order, performed substantially simultaneously, and performed in an opposite order.

The present disclosure can be implemented as a computer-readable code on a computer-readable recording medium and the computer-readable recording medium includes all types of recording devices for storing data that can be read by a computer system. Examples of the computer readable recording medium may include a ROM, a RAM, a CD-ROM, a magnetic tape, a floppy disk, an optical data storage device, and the like. Further, the computer readable recording media may be stored and executed as codes which may be distributed in the computer system connected through a network and read by a computer in a distribution method.

If it is not contrarily defined, all terms used herein have the same meanings as those generally understood by those skilled in the art. Terms which are defined in a generally used dictionary should be interpreted to have the same meaning as the meaning in the context of the related art, and are not interpreted as an ideal meaning or excessively formal meanings unless clearly defined in the present application.

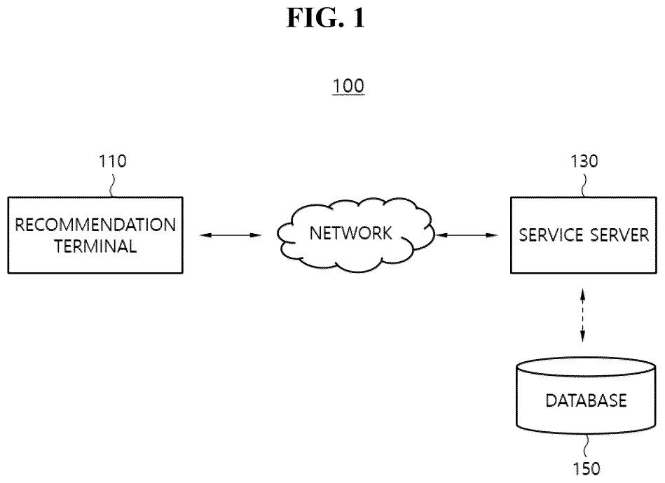

FIG. 1 is a diagram describing a recommendation service system according to the present disclosure.

Referring to FIG. 1, the recommendation service system 100 may include a recommendation terminal 110, a service server 130, and a database 150.

The recommendation terminal 110 may correspond to a terminal device that provides a golf club recommendation service according to the present disclosure, and is operated by a user. For example, the recommendation terminal 110 may be implemented to be included in a golf distance meter, but is, of course, not particularly limited thereto. The recommendation terminal 110 may be implemented to operate a manipulation of the user, and may measure a distance up to a ball or a distance between the ball and a target, and perform an operation of recommending a club suitable for the user as necessary.

The recommendation terminal 110 may be implemented as one device that constitutes the recommendation service system 100 according to the present disclosure, and the recommendation service system 100 may be implemented to be transformed to various types according to a purpose of a golf club recommendation service. Further, the recommendation terminal 110 may be implemented as a mobile terminal type which is enabled to operate in connection with the service server 130, but is not particularly limited thereto, and may be implemented as various devices including a golf electronic device or tablet PC. Meanwhile, the recommendation terminal 110 may be connected to the service server 130 through a network, and a plurality of recommendation terminals 110 may also be connected to the service server 130 at the same time.

The service server 130 may be implemented as a computer or a program that provides various functions and services required for a golf club recommendation service process according to the present disclosure. The service server 130 may be connected to the recommendation terminal 110 through a wired network or a wireless network such as Bluetooth, WiFi, or LTE, and may transmit and receive data to and from the recommendation terminal 110 through the network. Further, the service server 130 may be implemented to operate in connection with an independent external system (not illustrated in FIG. 1). For example, the service server 130 is connected to various screen golf courses or golf goods stores online to collect data regarding purchase of gold goods, and through this, may provide various functions and services required for the golf club recommendation service process according to the present disclosure.

The database 150 may correspond to a storage device that stores various information required during the operation of the service server 130. For example, the database 150 may store information on user of an indoor golf practice range for each user, or latest information regarding various golf clubs, but is not particularly limited thereto, and the service server 130 may store various types of information required for the golf club recommendation service process according to the present disclosure.

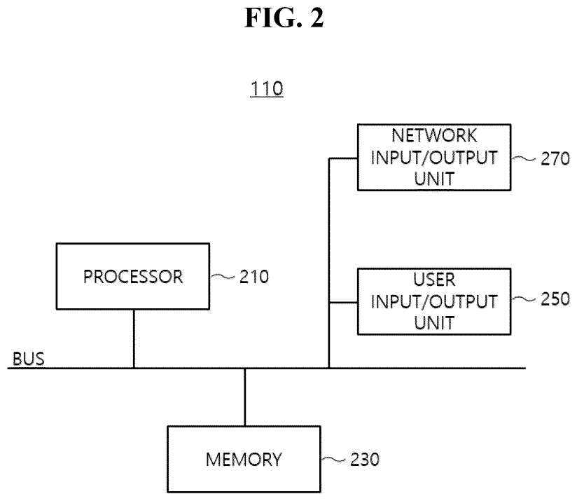

FIG. 2 is a diagram describing a system configuration of a recommendation terminal of FIG. 1.

Referring to FIG. 2, the recommendation terminal 110 may include a processor 210, a memory 230, a user input/output unit 250, and a network input/output unit 270.

The processor 210 may performs a procedure for performing a golf club recommendation service method according to the present disclosure, and manage a memory 230 read or written in such a process, and schedule a synchronization time between a volatile memory and a non-volatile memory in the memory 230. The processor 210 may control the overall operation of the recommendation terminal 110 and is electrically connected to the memory 230, the user input/output unit 250, and the network input/output unit 270 to control a data flow therebetween. The processor 210 may be implemented as a central processing unit (CPU) of the recommendation terminal 110, but is not particularly limited thereto, of course.

The memory 230 may include an auxiliary storage device implemented as the non-volatile memory such as a solid state disk (SSD) or a hard disk drive (HDD) and used to store all data required for the recommendation terminal 110, and may include a main storage device implemented as the volatile memory such as a Random Access Memory (RAM). In addition, the memory 230 may store a set of instructions that are executed by the electrically connected processor 210 to execute the golf club recommendation service method according to the present disclosure.

The user input/output unit 250 may include an environment for receiving a user input and an environment for outputting specific information to a user, and include an input device including an adapter such as, for example, a touch pad, a touch screen, an on-screen keyboard, or a pointing device, and an output device including an adapter such as a monitor or a touch screen. In an embodiment, the user input/output unit 250 may correspond to a computing device connected through a remote connection, and in such case, the recommendation terminal 110 may be performed as an independent server of the network to which the computing device is connected.

The network input/output unit 270 provides a communication environment for connection to another device through the network, and may include an adapter for communication such as, for example, Local Area Network (LAN), Metropolitan Area Network (MAN), Wide Area Network (WAN), and Value Added Network (VAN). Further, the network input/output unit 270 may be implemented to provide short-range communication functions such as WiFi and Bluetooth or wireless communication functions of 4G or higher for wireless transmission of data.

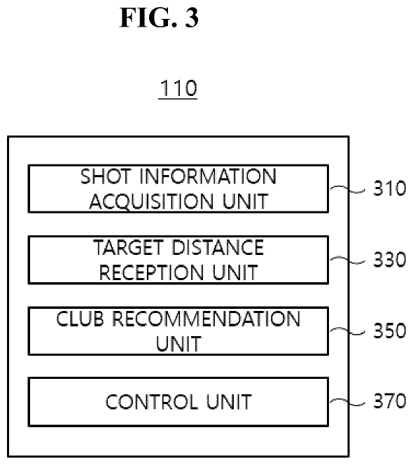

FIG. 3 is a diagram describing a functional configuration of the recommendation terminal of FIG. 1.

Referring to FIG. 3, the recommendation terminal 110 may perform the golf club recommendation service method according to the present disclosure. To this end, the recommendation terminal 110 may include a shot information acquisition unit 310, a target distance reception unit 330, a club recommendation unit 350, and a control unit 370.

At this time, the embodiment of the present disclosure does not particularly include all of the components at the same time, and according to respective embodiments, some of the components may be omitted, or the embodiment may also be implemented to selectively include some or all of the components. Hereinafter, the operation of each component will be described in detail.

The shot information acquisition unit 310 may acquire club-specific shot information including ball measurement data and club measurement data during a ball impact process of the user. To this end, the shot information acquisition unit 310 may operate in conjunction with a data collection device that monitors the ball impact process of the user, and collects the ball measurement data and the club measurement data. The data collection device may correspond to a dedicated device that may measure, collect, and store the ball measurement data and the club measurement data, and may be implemented as various types. Here, the club-specific shot information may include information regarding motions of the ball and the user which are uniquely measured according to a golf club which the user uses during the ball impact process. In an embodiment, the shot information acquisition unit 310 may collect ball impact time point information, location information, weather information, etc., jointly with the club-specific shot information.

In an embodiment, the shot information acquisition unit 310 may acquire club information of the user, and receive at least one of a ball speed, a launch angle, and a flight distance of the ball measured during the ball impact process as the ball measurement data. That is, the ball measurement data may correspond to detailed information regarding the motion of the ball according to an impact operation of the user. The ball speed may correspond to a motion speed of the ball by impact of the user, and may be measured as a unit of mile per hour, kilometer per hour, or meter per second measured at an impact point of the ball. The launch angle may correspond to a ballistic angle when the ball leaves a head face and starts flying into the air immediately after the impact of the user. The flight distance may correspond to a movement distance of the ball during a flight time, and correspond to a distance from the impact point of the ball to a point where the ball falls. The shot information acquisition unit 310 may collect one or more ball measurement data from the data collection device, and check data validity to eliminate invalid measurement data in advance as necessary.

In an embodiment, when at least some data of the ball speed, the launch angle, and the flight distance are not acquired, the shot information acquisition unit 310 may estimate some data not acquired with the remaining data. For example, the shot information acquisition unit 310 may estimate the flight distance of the ball by using the ball speed and the launch angle. The flight distance of the ball may increase as the ball speed is faster and the launch angle is closer to an optimal range, and the shot information acquisition unit 310 may derive a correlation between the ball speed, the launch angle, and the flight distance by analyzing pre-constructed ball measurement data, and then perform an operation for data estimation by using the derived correlation.

In an embodiment, the shot information acquisition unit 310 may acquire the club information of the user, and receive at least one of a club speed, a smash factor, and an attack angle of the club during the ball impact process as the club measurement data. The shot information acquisition unit 310 may receive the club information of the user directly from the user, and also automatically recognize the club information of the user through the data collection device as necessary. The shot information acquisition unit 310 may collect the club measurement data as a result of monitoring the impact operation of the user by interlocking with the data collection device. The shot information acquisition unit 310 may collect one or more club measurement data from the data collection device, and check data validity to eliminate invalid measurement data in advance as necessary.

Here, the club measurement data may correspond to detailed information regarding the motion of the club according to the impact operation of the user. The club speed may correspond to a speed at which a club head moves immediately before ball impact of the user. The smash factor may correspond to a value acquired by dividing the ball speed by a head speed, and correspond to a standard impact rate of the ball. The attack angle may correspond to data representing a vertical motion of the club upon the ball impact of the user, and may be expressed as a positive (+) or negative (−) value based on a point where an attack angle corresponding to a lowest point of swing is 0.

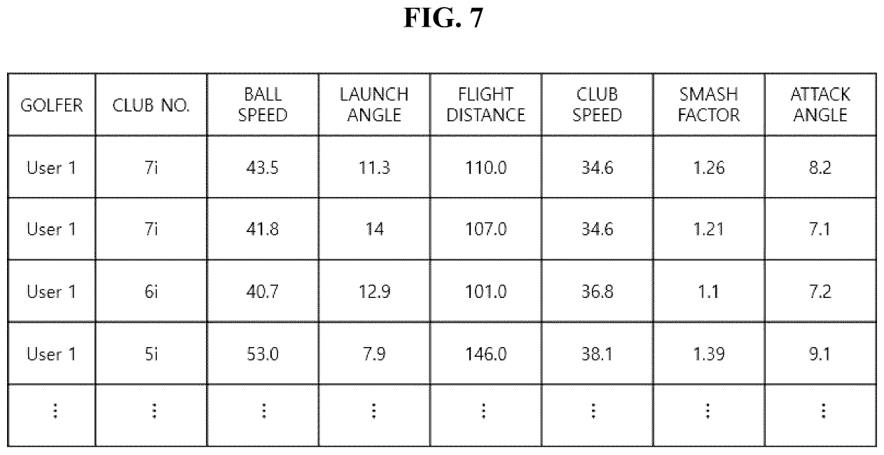

In an embodiment, the shot information acquisition unit 310 may load club-specific shot information measured when the user practices at the indoor golf practice range. To this end, the shot information acquisition unit 310 may operate in conjunction with a system of the indoor golf practice range. Further, the shot information acquisition unit 310 may operate by interlocking with the service server 130, and in this case, the service server 130 may be implemented to interlock with systems of various indoor golf practice ranges. For example, the shot information acquisition unit 310 may receive the club-specific shot information of the user collected through a golf simulator installed in the indoor golf practice range. In this case, the club-specific shot information may include a ball speed, a launch angle (a start angle of the ball), a carry (flight distance), a club speed, a smash factor (efficiency), and an attack angle (angle impact) according to a club number for each user (see FIG. 7).

The target distance reception unit 330 may receive a target distance constituted by horizontal and vertical distances from a current location up to a target location. Here, the current location may correspond to the location of the ball. To this end, the target distance reception unit 330 may operate in conjunction with a distance measurement module. At this time, the distance measurement module may include a laser transceiver. That is, the distance measurement module may measure a time when after a laser is sent to a target, the sent laser is reflected on and returned from the target, and multiplies the measure time and a speed of the laser to measure a distance up to the target. Meanwhile, the distance measurement module may also perform distance measurement by a GPS scheme. In an embodiment, the target distance reception unit 330 may perform a measurement operation for distances from the location of the user, i.e., a location of the recommendation terminal 110 up to the ball and the target.

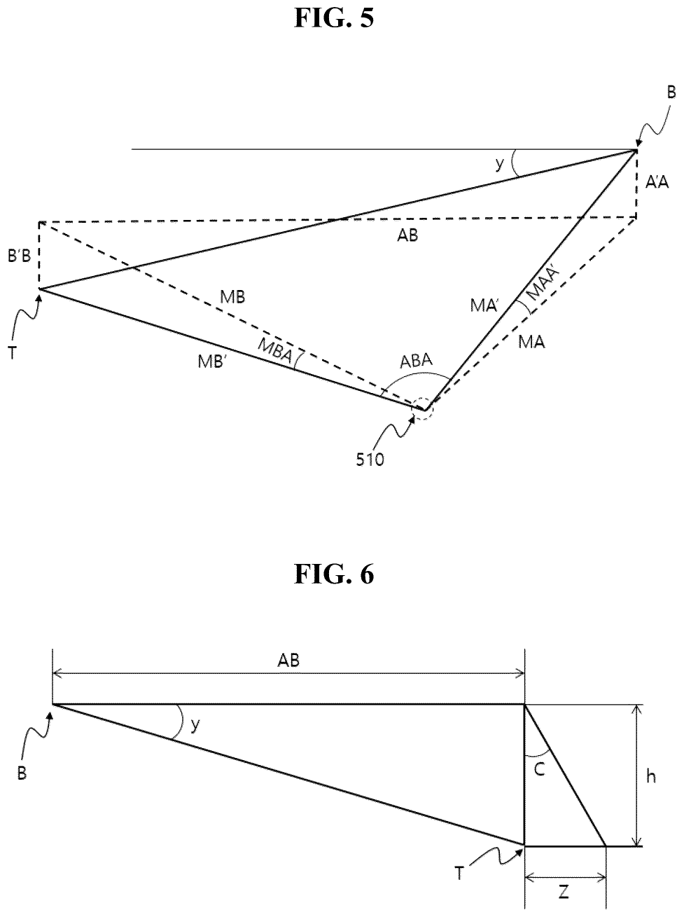

In an embodiment, the target distance reception unit 330 may calculate the horizontal and vertical distances based on measurement information including a straight distance and a slope up to the target location. The target distance reception unit 330 may measurer the straight distance up to the target location through the laser transceiver, and measure the slope (a slope angle) up to the target location through a gyro sensor. For example, when the distance measurement module which interlocks with the target distance reception unit 330 is disposed to face a specific target, the gyro sensor may measure an inclined angle of the distance measurement module as a slope angle from the current location up to the target based on a horizontal surface. Meanwhile, the distance measurement module may additionally include an acceleration sensor or a geomagnetic sensor in addition to the gyro sensor for measurement of various angles. A process of calculating the horizontal and vertical distances by the target distance reception unit 330 will be described in more detail through FIG. 5.

The club recommendation unit 350 may recommend a specific club by analyzing the club-specific shot information of the user based on the target distance. Even when the same club is used according to a club use scheme or an operation characteristic for each user, the users may have different ball flight distances, and the club recommendation unit 350 may recommend a club optimized to the user according to the target distance by analyzing a feature regarding club movement of the user. The club recommendation unit 350 may recommend one or more clubs for a specific target distance, and recommend information an impact method or a swing operation of the recommended club jointly with the club information.

In an embodiment, the club recommendation unit 350 may determine N (the N is a natural number) candidate clubs having a flight distance closest to the horizontal distance, and recommend one candidate club having a smallest measurement deviation of the club-specific shot information among N candidate clubs. The club recommendation unit 350 may select N candidate clubs by using a club-specific average flight distance based on the horizontal distance up to the target location. That is, the candidate club may correspond to the club selected based on the horizontal distance measured up to the target location. At this time, N which becomes a selection reference may be predetermined. The club recommendation unit 350 may select a club having a minimal measurement deviation of the shot information of each candidate club, and provide the selected club as a recommended club.

In an embodiment, the club recommendation unit 350 calculates an additional distance up to the target location based on a vertical distance from the ball up to the target to determine a shot recommendation distance based on the horizontal distance and the additional distance, and analyzes the club-specific shot information based on the shot recommendation distance to recommend a specific club. Here, the additional distance may correspond to an additional distance for sending the ball up to the target based on terrain information, and the shot recommendation distance may correspond to a calculated distance for club recommendation to which the horizontal distance and the additional distance are reflected. The club recommendation unit 350 may determine a club personalized to the user as the recommended club by matching the shot recommendation distance and the club-specific shot information.

For example, the club recommendation unit 350 may determine N (the N is a natural number) candidate clubs having a flight distance closest to the horizontal distance, and determine one candidate club having a smallest measurement deviation of the club-specific shot information among N candidate clubs as the recommended club. Meanwhile, the club recommendation unit 350 may determine the recommended club by applying various schemes according to the calculated shot recommendation distance in addition to the above scheme.

The control unit 370 may control an overall operation of the recommendation terminal 110, and manage a control flow or a data flow between the shot information acquisition unit 310, the target distance reception unit 330, and the club recommendation unit 350.

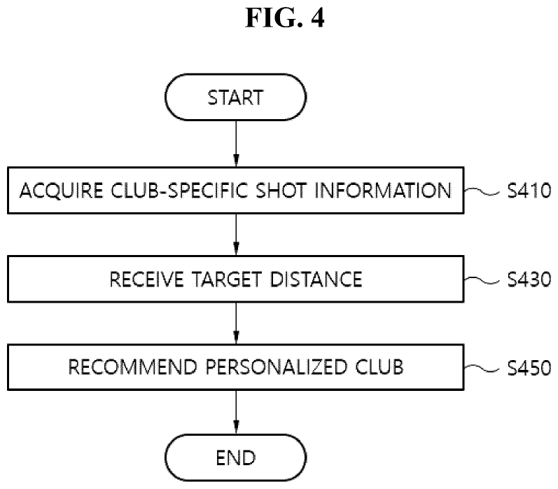

FIG. 4 is a flowchart describing an embodiment of a golf club recommendation service process according to the present disclosure.

Referring to FIG. 4, the recommendation terminal 110 may acquire club-specific shot information including ball measurement data and club measurement data during the ball impact process of the user (step S410). The recommendation terminal 110 may receive a target distance constituted by the horizontal and vertical distances from the current location up to the target location through the target distance reception unit 330 (step S430).

Further, the recommendation terminal 110 may recommend a specific club by analyzing the club-specific shot information of the user based on the target distance through the club recommendation unit 350 (step S450). In an embodiment, the club recommendation unit 350 may calculate a shot balance index of each club based on the club-specific shot information of the user, and recommend a specific club suitable for the target distance based on the shot balance index. Here, the shot balance index may correspond to individual suitability of the club which influences the flight distance of the ball according to a body shape and operation characteristics of the user. That is, a club having a higher shot balance index may correspond to a club more suitable for the user, and may provide a flight distance better than an average flight distance.

More specifically, the club recommendation unit 350 may calculate an influence index of each club measurement data according to a change amount of the flight distance for each club based on the club-specific shot information of the user. For example, the club recommendation unit 350 may normalize the change amount of the flight distance to a value within a specific range, and then calculate influence indexes of the club speed, the smash factor, and the attack angle, respectively.

Here, the influence index may correspond to quantifying a degree at which a change of the corresponding value is exerted to the change in flight distance. The club recommendation unit 350 may calculate a club-specific shot balance index of the user by using the influence index of each club measurement data. For example, the club-specific shot balance index may be calculated as a total sum or an average of the influence indexes of the respective club measurement data, but is not particularly limited thereto, of course.

Thereafter, the club recommendation unit 350 compares the shot balance index with one predetermined threshold or a plurality of thresholds determined stepwise to determine a specific interval distinguished by one or more thresholds to which the shot balance index belongs, and determine a recommended club matching the specific interval.

FIG. 5 is a diagram describing an embodiment of a process of calculating horizontal distances up to a ball and a target according to the present disclosure.

Referring to FIG. 5, the recommendation terminal 110 may perform a process of calculating the horizontal and vertical distances through the target distance reception unit 330. Specifically, the target distance reception unit 330 may sequentially perform a process of calculating a feature up to the ball, calculating a feature up to the target, and calculating a feature between the ball and the target.

When the user holds the recommendation terminal 510 and heads the recommendation terminal 510 to a golf ball B, the distance measurement module which interlocks with the target distance reception unit 330 of the recommendation terminal 510 may measure a straight distance MA′ up to the ball B. Further, the distance measurement module may measure a slope angle MAA up to the ball B based on the horizontal surface where the recommendation terminal 510 is positioned.

For example, in the case of FIG. 5, it may be assumed that MA′ is 48.531 m and MAA is 5.77 degrees. In this case, the distance measurement module may calculate a height A′A up to the ball and a horizontal distance MA up to the ball based on the horizontal surface where the recommendation terminal 510 is positioned by applying a triangular function method based on the MA′ value and MAA value. Specifically, the A′A and the MA may be calculated as in Equations 1 and 2 below, respectively.

$${A}^{′} A = M {A}^{′} \cdot sin M M A = 48.531 m \cdot sin 5.77 ° = 4.878 m \qquad [ Equation 1 ]$$

$$M A = M {A}^{′} \cdot cos M M A = 48.531 m \cdot cos 5.77 ° = 48.29 m \qquad [ Equation 2 ]$$

Meanwhile, when the user holds the recommendation terminal 510 and heads the recommendation terminal 510 to a target T, the distance measurement module which interlocks with the target distance reception unit 330 of the recommendation terminal 510 may measure a straight distance MB′ up to the target T. Further, the distance measurement module may measure a slope angle MBA up to the target T based on the horizontal surface where the recommendation terminal 510 is positioned.

For example, in the case of FIG. 5, it may be assumed that MB′ is 73.99 m and MAA is-3.10 degrees. A case where MAA is negative number may indicate that the target T is positioned in a lower direction by 3.10 degrees based on the horizontal surface.

Meanwhile, when the recommendation terminal 510 according to the embodiment of the present disclosure performs distance measurement in a GPS based distance measurement scheme or a complex scheme of a GPS and a laser, the distance measurement module that interlocks with the target distance reception unit 330 may acquire absolute location information (e.g., information including latitude, longitude, altitude, etc.) of the ball B.

For example, a current location of the recommendation terminal 510 may be measured through a GPS satellite signal, and a location of the ball B from the current location may be calculated based on Equations 1 and 2 above, so an absolute location of the ball B may be measured through the calculated location.

The distance measurement module may calculate a height B′B up to the target and a horizontal distance MB up to the target based on the horizontal surface where the recommendation terminal 510 is positioned by applying the triangular function method based on the MB′ value and MBA value. Specifically, the B′B and the MB may be calculated as in Equations 3 and 4 below, respectively.

$${B}^{′} B = M {B}^{′} \cdot sin M B A = 73.99 m \cdot sin ( - 3.1 ° ) = - 4. m \qquad [ Equation 3 ]$$

$$M B = M {B}^{′} \cdot cos M B A = 73.99 m \cdot cos ( - 3.1 ° ) = 73.88 m \qquad [ Equation 4 ]$$

Meanwhile, when the user holds the recommendation terminal 510 and heads the recommendation terminal 510 to the ball B, and then heads the recommendation terminal 510 to the target T, the distance measurement module which interlocks with the target distance reception unit 330 may measure horizontal angles ABA of the ball B and the target T based on the location of the recommendation terminal 510. For example, in the case of FIG. 5, it may be assumed that ABA is 90.23 degrees.

Meanwhile, when the recommendation terminal 510 according to the embodiment of the present disclosure performs the distance measurement in the GPS based distance measurement scheme or the complex scheme of the GPS and the laser, the distance measurement module that interlocks with the target distance reception unit 330 may acquire absolute location information (e.g., information including latitude, longitude, altitude, etc.) of the target T.

For example, the current location of the recommendation terminal 510 may be measured through the GPS satellite signal, and a location of the target T from the current location may be calculated based on Equations 3 and 4 above, so an absolute location of the target T may be measured through the calculated location.

Further, hole information of each golf course may be stored in the service server 130 or the database 150, and since the corresponding information stores location information of each hole-specific target T, the recommendation terminal 510 receives the corresponding information from the service server 130 or the database 150 to acquire the absolute location of the target T.

In an embodiment, the recommendation terminal 510 may calculate horizontal distances AB up to the ball B and the target T as in Equation 5 below based on a horizontal distance MA from the location of the recommendation terminal 510 up to the ball B, a horizontal distance MB up to the target T, and a horizontal angle ABA between the ball B and the target T through the distance measurement module which interlocks with the target distance reception unit 330.

$$[ Equation 5 ]$$

$$A B = \sqrt{M {A}^{2} + M {B}^{2} - 2 \cdot M A \cdot M B \cdot cos A B A} = \sqrt{{( 48.29 m )}^{2} + {( 73.88 m )}^{2} - 2 \cdot ( 48.29 m ) \cdot ( 73.88 m ) \cdot cos 90.23 °} = 88.422 m$$

Meanwhile, a height from the ball B up to the target T based on a height A′A from a current point up to the ball B, and a height B′B up to the target T (B′B−A′A), and when the corresponding value, and the horizontal distance AB(x) up to the ball B and the target T calculated above are used, a slope angle y up to the target T based on the ball B may be calculated as in Equation 6 below.

$$y = a tan \frac{{B}^{′} B - {A}^{′} A}{A B} = a tan \frac{- 4. - 4.878}{88.422} = - 5.733 ° \qquad [ Equation 6 ]$$

FIG. 6 is a diagram describing an embodiment of a process of calculating a shot recommendation distance for sending the ball to the target according to the present disclosure.

Referring to FIG. 6, the recommendation terminal 110 may calculate the shot recommendation distance from the ball B up to the target T based on the horizontal distances AB up to the ball B and the target T through the target distance reception unit 330.

First, the user may input reference information through the recommendation terminal 110 or a separate device which is enabled to communicate therewith, for example, a smartphone. Here, the reference information may correspond to information for determining an angle between a direction in which the golf ball falls to the ground after the golfer shots the ball, and a vertical line, i.e., a landing angle, and the landing angle may be used for a process of calculating a recommendation distance of the shot for sending the golf ball up to the target.

For example, the reference information may correspond to a distance at which the golf ball may be sent when performing the shot by using Iron #7 7i. The reference information may also be input directly by the user, but may also be determined according to predetermined information (e.g., 140 m or 150 m). That is, a matching table between the reference information and the landing angle may be pre-constructed, and then used.

The recommendation terminal 110 may calculate a recommended distance to perform the shot in order to send the ball up to the target by using a height h from the ball B up to the target T, and a landing angle acquired by referring to the reference information through the target distance reception unit 330. According to a result of already calculating the horizontal distance AB from the ball B up to the target T, only an additional distance Z for sending the ball B up to the target T may be calculated, and expressed as in Equation 7 below.

$$Z = h \cdot tan C = ( {B}^{′} B - {A}^{′} A ) \cdot tan C \qquad [ Equation 7 ]$$

Here, h represents the height from the ball B up to the target T, and this may be calculated based on the height A′A from the current location up to the ball B and the height B′B up to the target T from the current location which are already calculated (That is, h=B′B-A′A=−4.0 m-4.8 m=−8.8 m). For example, when the landing angle C is 41 degrees, the additional distance Z may become −7.7 m.

Meanwhile, when the recommendation terminal 110 according to the embodiment of the present disclosure performs the distance measurement in the GPS based distance measurement scheme or the complex scheme of the GPS and the laser, the distance measurement module which interlocks with the target distance reception unit 330 may calculate the straight distance, the horizontal distance, and the height from the ball B up to the target T by using the absolute location information of the ball B and the target T as described above, and calculate the additional distance Z and the shot recommendation distance by the same scheme as described above.

The present disclosure has been described with reference to the preferred embodiments of the present disclosure, but those skilled in the art will understand that the present disclosure can be variously modified and changed without departing from the spirit and the scope of the present disclosure which are defined in the appended claims.

### EXPLANATION OF REFERENCE NUMERALS AND SYMBOLS

100: Recommendation service system
110, 510: Recommendation terminal
130: Service server
150: Database
210: Processor
230: Memory,
250: User input/output unit
270: Network input/output unit
310: Shot information acquisition unit
330: Target distance reception unit
350: Club recommendation unit
370: Control unit

1. A golf club recommendation service device comprising:
a shot information acquisition unit acquiring club-specific shot information including ball measurement data and club measurement data during a ball impact process of a user;
a target distance reception unit receiving a target distance constituted by horizontal and vertical distances from a current location up to a target location; and
a club recommendation unit recommending a specific club by analyzing the club-specific shot information of the user based on the target distance.

2. The golf club recommendation service device of claim 1, wherein the shot information acquisition unit acquires club information of the user, and receives at least one of a ball speed, a launch angle, and a flight distance of a ball measured during the ball impact process as the ball measurement data.

3. The golf club recommendation service device of claim 2, wherein when at least some data of the ball speed, the launch angle, and the flight distance are not acquired, the shot information acquisition unit estimates some data not acquired with the remaining data.

4. The golf club recommendation service device of claim 1, wherein the shot information acquisition unit acquires the club information of the user, and receives at least one of a club speed, a smash factor, and an attack angle of a club ball measured during the ball impact process as the club measurement data.

5. The golf club recommendation service device of claim 1, wherein the shot information acquisition unit loads the club-specific shot information measured when the user practices in an indoor golf practice range.

6. The golf club recommendation service device of claim 1, wherein the target distance reception unit calculates the horizontal and vertical distances based on measurement information including a straight distance and a slope up to the target location.

7. The golf club recommendation service device of claim 1, wherein the club recommendation unit determines N (the N is a natural number) candidate clubs having a flight distance closest to the horizontal distance, and recommends one candidate club having a smallest measurement deviation of the club-specific shot information among the N candidate clubs.

8. The golf club recommendation service device of claim 1, wherein the club recommendation unit calculates an additional distance up to the target location based on the vertical distance to determine a shot recommendation distance based on the horizontal distance and the additional distance, and analyzes the club-specific shot information based on the shot recommendation distance to recommend a specific club.
